(19) United States
(12) Patent Application Publication
(10) Pub. No.: US 2019/0029539 A1
(43) Pub. Date: Jan. 31, 2019

(54) PHYSICAL SIGN DETECTING EARPHONE AND PHYSICAL SIGN DETECTING METHOD

(71) Applicant: BOE TECHNOLOGY GROUP CO., LTD., Beijing (CN)

(72) Inventor: Hui DU, Beijing (CN)

(21) Appl. No.: 15/942,816

(22) Filed: Apr. 2, 2018

(30) Foreign Application Priority Data
Jul. 28, 2017 (CN) 201710633308.3

(51) Int. Cl. A61B 5/021; A61B 5/16; A61B 5/00; H04R 1/10

(52) U.S. Cl. CPC A61B 5/02125; A61B 5/165; H04R 1/1016; A61B 5/7278; H04R 1/1091; A61B 5/6803

(57) ABSTRACT

A physical sign detecting earphone and a physical sign detecting method are disclosed. The physical sign detecting earphone includes a processor, and photoelectric sensors configured in left and right receivers respectively. The photoelectric sensors are used for collecting two pulse signals of left and right ears. The processor is used for performing physical sign detecting based on the pulse signals collected by the photoelectric sensors to obtain physical sign information. The embodiments of the disclosure solve the problem that the blood pressure measurement and fatigue detection in the prior art have poor convenience and measurement cannot be performed in real time.

### CROSS REFERENCE OF RELATED APPLICATION

The present application claims the priority of the Chinese patent application No. 201710633308.3 filed on Jul. 31, 2017, which is entirely incorporated herein by reference.

### TECHNICAL FIELD

The disclosure relates to the technical field of physical sign detecting, particularly to a physical sign detecting earphone and a physical sign detecting method.

### BACKGROUND

With continuous development of the computer technology, there are more and more miniaturized components which provide a possibility for implementing intelligent wearable devices. The miniaturized sensors can extract user physiological parameters, and at the same time, provide the possibility of integrating sensors into different products.

As the pace of life continues to speed up, high blood pressure has become a major hidden danger that induces disease. In addition, most of people have different levels of mental fatigue. If the mental fatigue cannot be found and released timely, it will cause disease and be harmful to people's health. Blood pressure measurement devices in the prior art mainly include mercury blood pressure measurement devices and electronic blood pressure measurement devices. They require users to wear a cuff or wristband when measuring, and require the users to initiate measurement. They provide a less comfort measurement, a complex measuring way and an inconvenient use. The fatigue detection makes determination mainly based on a computer, which has problems, such as the detection being, the detection being not able to be performed in real time, and so on. The blood pressure measurement and fatigue detection in the prior art both have to make measurements by particular devices, e.g., a blood pressure measurement device, a computer terminal etc. These devices have problems of inconvenient carry and non-real-time measurement.

To sum up, the blood pressure measurement and fatigue detection in the prior art have to be performed by particular devices which are not easy to carry and have a complex measuring way. Therefore, they have problems of poor convenience and non-real-time measurement.

### SUMMARY

In order to solve the above technical problem, embodiments of the disclosure provide a physical sign detecting earphone and a physical sign detecting method.

According to one aspect of the disclosure, a physical sign detecting earphone is provided. The physical sign detecting earphone may comprise: a processor, and photoelectric sensors configured in left and right receivers respectively. The photoelectric sensors are used for collecting two pulse signals of left and right ears. The processor is used for performing physical sign detecting based on the pulse signals collected by the photoelectric sensors to obtain physical sign information.

In an embodiment, the physical sign information comprises: one or more of a blood pressure value and a fatigue degree.

In an embodiment, the processor performing physical sign detecting to obtain a blood pressure value may comprise: calculating a pulse transmission time based on the two pulse signals collected by the photoelectric sensors, the pulse transmission time being a time difference between peak values of the two pulse signals within a same electrocardio cycle; and calculating the blood pressure value based on a preconfigured blood pressure model and the pulse transmission time.

In an embodiment, the preconfigured blood pressure model comprises a formula:

$$BP = \frac{1}{γ} [ ln ( \frac{ρ {dS}^{2}}{α {E}_{0}} ) - 2 ln PTT ] ;$$

wherein the BP represents the blood pressure value, the PTT represents the pulse transmission time, the γ represents an elastic coefficient of a blood vessel, the ρ represents blood density, the d represents a diameter of the blood vessel, the S² represents a cross-sectional area of the blood vessel, the α represents a thickness of the blood vessel, the E₀ represents an initial vascular elasticity modulus.

In an embodiment, the photoelectric sensors are used for collecting two pulse signals of left and right ears in a non-fatigue state of a user when the physical sign detecting earphone is initially used, and collecting two pulse signals of left and right ears in real time. The processor is used for calculating a pulse transmission time in the non-fatigue state based on the pulse signals in the non-fatigue state collected by the photoelectric sensors, calculating a pulse transmission time based on the two pulse signals connected by the photoelectric sensors in real time, and calculating a real time blood pressure value through a formula:

$$Δ BP = - \frac{2}{γ PPT} Δ PTT .$$

In this formula, the PTT represents a pulse transmission time measured in real time, the ΔBP represents a difference value between a blood pressure value in the non-fatigue state and a blood pressure value measured in real time, the ΔPTT represents a difference value between the pulse transmission time in the non-fatigue state and the pulse transmission time measured in real time, the γ represents an elastic coefficient of a blood vessel, the blood pressure value in the non-fatigue state is measured by a blood pressure measurement device.

In an embodiment, the processor performing physical sign detecting to obtain a fatigue degree comprises: calculating a pulse peak-to-peak time period based on a pulse signal connected by a photoelectric sensor in one of the left receiver and the right receiver; performing frequency spectrum analysis to a pulse signal within the pulse peak-to-peak time period to obtain frequency spectrum parameters; and determining the fatigue degree based on the frequency spectrum parameters.

In an embodiment, the frequency spectrum parameters comprise: a low frequency power, a high frequency power and a ratio of the low frequency power to the high frequency power. Determining the fatigue degree based on the frequency spectrum parameters may comprise: compared to standard values of the frequency spectrum parameters, when the low frequency power increases, the ratio of the low frequency power to the high frequency power increases, and the high frequency power decreases, determining that the fatigue degree increases; or, when the low frequency power increases, the ratio of the low frequency power to the high frequency power increases, and the high frequency power is constant, determining that the fatigue degree increases; or, when the low frequency power is constant, the ratio of the low frequency power to the high frequency power increases, and the high frequency power decreases, determining that the fatigue degree increases.

In an embodiment, the standard values of the frequency spectrum parameters are obtained by: the photoelectric sensor in one of the left receiver and the right receiver collecting a pulse signal in the non-fatigue state; and the processor calculating a pulse peak-to-peak time period in the non-fatigue state based on the pulse signal in the non-fatigue state collected by the photoelectric sensor, and performing frequency spectrum analysis to the pulse signal within the pulse peak-to-peak time period to obtain values of frequency spectrum parameters as the standard values of the frequency spectrum parameters.

In an embodiment, the physical sign detecting earphone can further comprise: a prompt module for prompting an abnormal state of the physical sign information when the physical sign information obtained by the processor is not conformity with a preset condition.

In an embodiment, the physical sign information being not conformity with a preset condition comprises one or more of: the blood pressure value being out of a preset blood pressure threshold range; and a time in which a user is in a fatigue state being longer than a preset time threshold.

In an embodiment, the physical sign detecting earphone can further comprise: a communication module for transmitting the physical sign information to a terminal.

According to another aspect of the disclosure, a physical sign detecting method is provided. The physical sign detecting method can comprise: collecting two pulse signals of left and right ears by photoelectric sensors configured in left and right receivers of a physical sign detecting earphone respectively; and performing physical sign detecting based on the pulse signals to obtain physical sign information.

In an embodiment, the physical sign information comprises: one or more of a blood pressure value and a fatigue degree.

In an embodiment, performing physical sign detecting based on the pulse signals to obtain a blood pressure value may comprise: calculating a pulse transmission time based on the two pulse signals collected by the photoelectric sensors, the pulse transmission time being a time difference between peak values of the two pulse signals within a same electrocardio cycle; and calculating the blood pressure value based on a preconfigured blood pressure model and the pulse transmission time.

In an embodiment, the preconfigured blood pressure model comprises a formula:

$$BP = \frac{1}{γ} [ ln ( \frac{ρ {dS}^{2}}{α {E}_{0}} ) - 2 ln PTT ] ;$$

wherein the BP represents the blood pressure value, the PTT represents the pulse transmission time, the γ represents an elastic coefficient of a blood vessel, the ρ represents blood density, the d represents a diameter of the blood vessel, the S² represents a cross-sectional area of the blood vessel, the α represents a thickness of the blood vessel, the E₀ represents an initial vascular elasticity modulus.

In an embodiment, the physical sign detecting method can further comprise: collecting two pulse signals of left and right ears in a non-fatigue state of a user by the photoelectric sensors when the physical sign detecting earphone is initially used; calculating a pulse transmission time in the non-fatigue state based on the two pulse signals in the non-fatigue state; collecting two pulse signals of left and right ears by the photoelectric sensors in real time; calculating a pulse transmission time based on the two pulse signals connected by the photoelectric sensors in real time, and calculating a real time blood pressure value through a formula:

$$Δ BP = - \frac{2}{γ PPT} Δ PTT ;$$

wherein, the PTT represents a pulse transmission time measured in real time, the ΔBP represents a difference value between a blood pressure value in the non-fatigue state and a blood pressure value measured in real time, the ΔPTT represents a difference value between the pulse transmission time in the non-fatigue state and the pulse transmission time measured in real time, the γ represents an elastic coefficient of a blood vessel, the blood pressure value in the non-fatigue state is measured by a blood pressure measurement device.

In an embodiment, performing physical sign detecting based on the pulse signals to obtain a fatigue degree can comprise: calculating a pulse peak-to-peak time period based on a pulse signal connected by a photoelectric sensor in one of the left receiver and the right receiver; performing frequency spectrum analysis to a pulse signal within the pulse peak-to-peak time period to obtain frequency spectrum parameters; and, determining the fatigue degree based on the frequency spectrum parameters.

In an embodiment, the frequency spectrum parameters can comprise: a low frequency power, a high frequency power and a ratio of the low frequency power to the high frequency power. Determining the fatigue degree based on the frequency spectrum parameters can comprise: compared to standard values of the frequency spectrum parameters, when the low frequency power increases, the ratio of the low frequency power to the high frequency power increases, and the high frequency power decreases, determining that the fatigue degree increases; or, when the low frequency power increases, the ratio of the low frequency power to the high frequency power increases, and the high frequency power is constant, determining that the fatigue degree increases; or, when the low frequency power is constant, the ratio of the low frequency power to the high frequency power increases, and the high frequency power decreases, determining that the fatigue degree increases.

In an embodiment, the physical sign detecting method can further comprise: prompting an abnormal state of the physical sign information when the obtained physical sign information is not conformity with a preset condition. The physical sign information being not conformity with a preset condition can comprise one or more of: the blood pressure value being out of a preset blood pressure threshold range; and a time in which a user is in a fatigue state being longer than a preset time threshold.

In an embodiment, the physical sign detecting method can further comprise: transmitting the physical sign information to a terminal.

### BRIEF DESCRIPTION OF THE DRAWINGS

The drawings are used for providing further understanding to the technical solutions of the disclosure and form a portion of the Specification to explain the technical solutions of the disclosure together with embodiments of the present application, and are not limitations to the technical solutions of the disclosure.

FIG. 1 is a schematic diagram of a module architecture of a physical sign detecting earphone provided by an embodiment of the disclosure;

FIG. 2 is a schematic diagram of a physical sign detecting earphone provided by an embodiment of the disclosure;

FIG. 3 is a waveform diagram of transmission of pulse signals detected in a physical sign detecting earphone provided by an embodiment of the disclosure;

FIG. 4 is a fitting curve diagram obtained based on a measured blood pressure value and pulse transmission time;

FIG. 5 is an example diagram of a power spectrum of a pulse signal within a pulse peak-to-peak time period;

FIG. 6 is a schematic diagram of a measurement principle of a physical sign detecting earphone provided by an embodiment of the disclosure;

FIG. 7 is a schematic diagram of a module architecture of a physical sign detecting earphone provided by another embodiment of the disclosure;

FIG. 8 is a flow chart of a physical sign detecting method provided by an embodiment of the disclosure;

FIG. 9 is a flow chart of a physical sign detecting method provided by another embodiment of the disclosure; and

FIG. 10 is a flow chart of a physical sign detecting method provided by a yet another embodiment of the disclosure.

### EMBODIMENTS

In order to enable objects, technical solutions and advantages of the disclosure to be more clear, embodiments of the disclosure will be explained below in detail in conjunction with the drawings. It should be noted that in the case of not conflicting, the embodiments in the present application and the features in the embodiments can be arbitrarily combined with each other.

The several following specific embodiments provided by the disclosure can be combined with each other. The same or similar concepts or processes may not be repeated in some embodiments.

The embodiments of the disclosure provide a physical sign detecting earphone and a physical sign detecting method. The physical sign detecting earphone collects two pulse signals of left and right ears by photoelectric sensors configured in left and right receivers, and performs physical sign detecting based on the collected pulse signals by a processor, to obtain physical sign information which can reflect a physical state of a user of the earphone. The physical sign detecting earphone provided by the embodiment of the disclosure is easy to carry and can perform measurement of the physical sign information when the user uses the earphone, thereby achieving the object of measuring the physical sign information of the user in real time, and solving the problems that the blood pressure measurement and fatigue detection in the prior art have poor convenience and cannot be performed in real time.

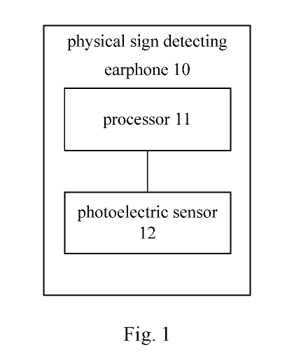

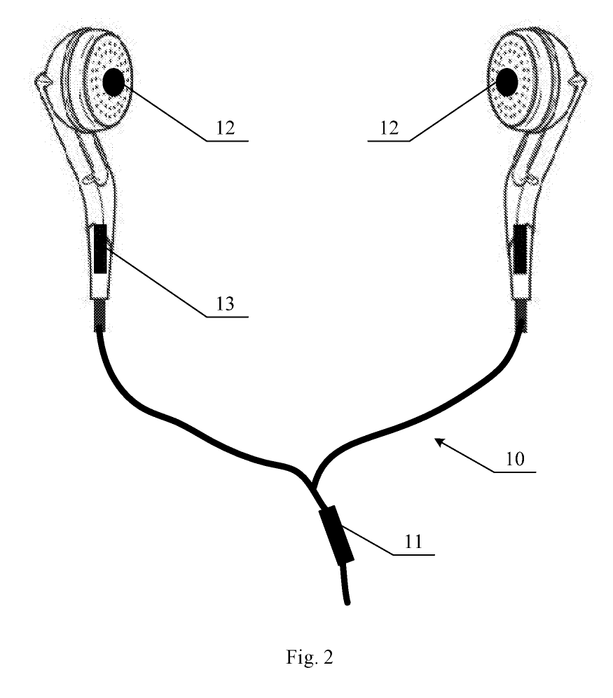

FIG. 1 is a schematic diagram of a module architecture of a physical sign detecting earphone provided by an embodiment of the disclosure. FIG. 2 is a schematic diagram of a physical sign detecting earphone provided by an embodiment of the disclosure. As shown in FIGS. 1 and 2, the physical sign detecting earphone 10 provided by an embodiment of the disclosure can comprise: a processor 11, and photoelectric sensors 12 configured in left and right receivers respectively. The photoelectric sensors 12 are used for collecting two pulse signals of left and right ears. The processor 11 is used for performing physical sign detecting based on the pulse signals collected by the photoelectric sensors 12 to obtain physical information.

The physical sign detecting earphone 10 provided by the embodiment of the disclosure can be used for performing intelligent measurement to the physical sign information of a human body. With popularity of terminal devices such as an intelligent phone, the earphone has become a terminal accessory that is widely used. Hence, the embodiment of the disclosure integrates a physical sign detecting function into an earphone and designs the physical sign detecting earphone 10. The physical sign detecting earphone 10 in the embodiment of the disclosure for example can be an in-ear earphone or earphone mic configured for a terminal device which for example can be an intelligent phone, a tablet computer or a personal digital assistant (PDA) etc. FIG. 2 is a schematic diagram of a physical sign detecting earphone. The photoelectric sensors 12 can be arranged in the left and right receivers of the earphone respectively, and the processor 11 connected with the photoelectric sensors 12 in the left and right receivers respectively can be arranged in the earphone. The photoelectric sensors 12 for example are reflective photoelectric sensor 12. In this way, when a user uses the physical sign detecting earphone 10 to listen to music or watch videos, the physical sign detecting earphone 10 can respectively collect the pulse signals of the left and right ears of the user through the photoelectric sensors 12 arranged in the left and right receivers. As such, the physical sign detecting earphone 10 can collect the two pulse signals. In an embodiment, the photoelectric sensors 12 can also perform to the two collected pulse signals preprocessing operations such as signal amplification, analog-to-digital conversion and noise elimination, and so on. Subsequently, the two collected or preprocessed pulse signals are transmitted to the processor 11 for performing physical sign detecting.

In an embodiment of the disclosure, the processor 11 can perform physical sign detecting based on the two pulse signals, after receiving the two pulse signals transmitted by the photoelectric sensors 12. Because the two pulse signals are pulse signals collected at different positions of the human body, and distances between the different positions of the human body and a heart are different, transmission of the two pulse signals have different wave peaks and wave valleys. The physical sign information of the user can be calculated based on a difference between the two pulse signals. The physical sign information can reflect the physical state of the user.

In an embodiment of the disclosure, the physical sign information obtained by the processor 11 can include but not limited to: one or more of a blood pressure value and a fatigue degree. In the current situation that the pace of life continues to speed up, a high blood pressure has become a major hidden danger that induces people's diseases and has severely harmed people's health. In addition, under the high pace life and high intensity work, most of people has different levels of mental fatigue, which will also induce some diseases and harm people's health. Therefore, the physical sign information in the embodiments of the disclosure can be used for monitoring whether a user has a high blood pressure and whether a user is in the state of mental fatigue. It should be noted that the physical sign information in the embodiments of the disclosure can also include other indexes such as a heart rate value that can reflect a physical state of a user.

The blood pressure measurement method in the prior art generally employs a blood pressure measurement device which mainly includes a mercury blood pressure measurement device and an electronic blood pressure measurement device. They require a user to wear a cuff or wristband when measuring and require the user to perform measurement initiatively. They provide a less comfort measurement, a complex measuring way and an inconvenient use. The fatigue detection makes determination mainly based on the computer, which has the problems of inconvenient detection and non-in-time detection, etc. The blood pressure measurement and fatigue detection in the prior art both have to be performed by particular devices, e.g., a blood pressure measurement device, a computer terminal etc. that have problems of inconvenient carry and non-in-time measurement.

Because the physical sign detecting earphone 10 in the embodiment of the disclosure is provided with photoelectric sensors 12, compared to ways of measuring the blood pressure value and the fatigue degree in the prior art, when a user uses the earphone to listen to songs or watch videos, the physical sign detecting earphone 10 can collect two pulse signals of left and right ears of the user in real time, and can process the collected pulse signals, so as to obtain physical sign information that can reflect the physical state of the user. By means of the physical sign detecting earphone 10 provided by the embodiment of the disclosure, in the process of obtaining the physical sign information, the physical sign information of the user does not have to be measured by a particular device, and instead can be obtained directly by the earphone, which improves comfort degree in the physical sign detecting process, is easy to use and achieves the object of measuring the physical sign information in real time.

The physical sign detecting earphone provided by an embodiment of the disclosure collects two pulse signals of left and right ears by photoelectric sensors configured in left and right receivers and performs physical sign detecting by a processor based on the collected pulse signals so as to obtain the physical sign information. The physical sign information can reflect the physical state of a user of the earphone. The physical sign detecting earphone provided by an embodiment of the disclosure is easy to carry and can perform measurement of the physical sign information when the user uses the earphone, thereby achieving the object of measuring the physical sign information of the user in real time and solving the problems that the blood pressure measurement and fatigue detection in the prior art have poor convenience and cannot be performed in real time.

The above embodiments of the disclosure have shown that the physical sign information can include: one or more of a blood pressure value and a fatigue degree.

In an embodiment of the disclosure, implementation for performing physical sign detecting by the processor 11 to obtain the blood pressure value can comprise: calculating a pulse transmission time based on two pulse signals collected by photoelectric sensors 12, the pulse transmission time being a time difference between peak values of the two pulse signals within a same electrocardio cycle; and calculating the blood pressure value based on a preconfigured blood pressure model and the pulse transmission time.

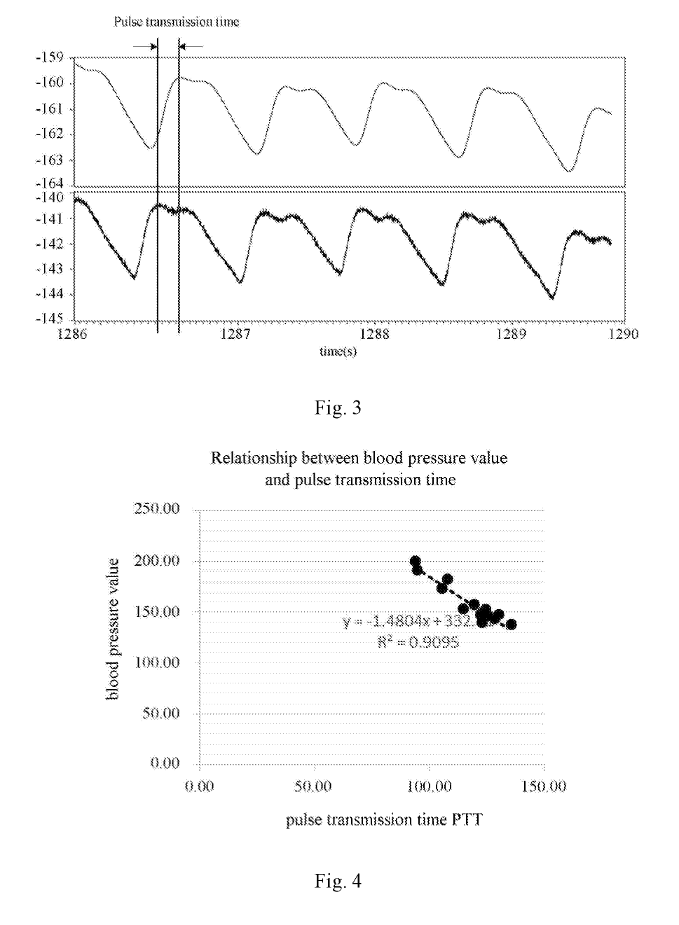

In an embodiment of the disclosure, in the process of measuring the blood pressure value, it is required to firstly calculate the time difference between the peak values of two pulse signals within the same electrocardio cycle, i.e., the pulse transmission time. FIG. 3 is an example waveform diagram of transmission of pulse signals detected in a physical sign detecting earphone provided by an embodiment of the disclosure. The upper waveform in FIG. 3 is the collected pulse signal of the left ear, and the lower waveform is the collected pulse signal of the right ear. Because body parts from which the pulse signals are collected by the photoelectric sensors 12 in the left and right receivers are different, times of reaching the detected parts from cardiac ejection will be different, i.e., the wave peaks and the wave valleys of the two collected pulse signals are different. The pulse transmission time can be obtained by comparing the time difference between the pulse wave peaks of the two pulse signals within the same electrocardio cycle. Subsequently, a blood pressure value corresponding to the collected pulse signals, i.e., a current blood pressure value of a user, can be calculated by substituting the obtained pulse transmission time into a preconfigured blood pressure model.

According to an embodiment of the disclosure, the preconfigured blood pressure model can be:

$$BP = \frac{1}{γ} [ ln ( \frac{ρ {dS}^{2}}{α {E}_{0}} ) - 2 ln PTT ] ; \qquad ( 1 )$$

In the above formula (1), BP represents a blood pressure value, PTT represents a pulse transmission time, γ represents an elastic coefficient of a blood vessel, ρ represents blood density, d represents a diameter of the blood vessel, S² represents a cross-sectional area of the blood vessel, α represents a thickness of the blood vessel, E₀ represents an initial vascular elasticity modulus. According to the disclosure, the parameters γ, ρ, d, S², α, E₀ can be typical constants obtained by sampling a large number of people or performing statistical calculation to a large amount of data. In practice, with continuous increase of the sampling and the data, values of these parameters can be continuously kept accurate, so as to enable the blood pressure value calculated based on the formula (1) to be more accurate.

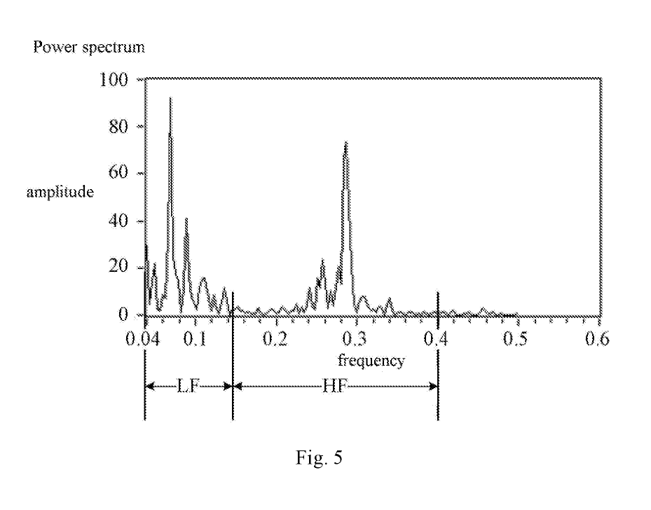

FIG. 4 is a fitting curve diagram obtained based on the measured exemplary blood pressure value and pulse transmission time. In FIG. 4, the longitudinal coordinate is the blood pressure value, and the horizontal coordinate is the pulse transmission time. The blackspots in FIG. 4 are experimental data obtained by performing measurement to the same user for many times wherein the blood pressure value is obtained through e.g., a mercury blood pressure measurement device, and the pulse transmission time is obtained based on the method as described above herein. The function y in FIG. 4 is obtained by fitting of these experimental data and represents the blood pressure value. x represents the pulse transmission time. It can be seen from FIG. 4 that the blood pressure value and the pulse transmission time have a good linear relationship, and a correlation coefficient R² is up to 0.9095. This also proves that, according to the disclosure, it is feasible to calculate the blood pressure value using the pulse transmission time. The function y in FIG. 4 may be only suitable for a particular user since the data comes from the particular user. However, according to the disclosure, more users and more measurement data can be fitted to modify the coefficient of the function y, so as to obtain a general function y that is suitable for a large amount of users. As such, in the case of that the pulse transmission time is known, the blood pressure value can be calculated based on the function y.

In an embodiment of the disclosure, implementation for performing physical sign detecting by the processor 11 to obtain a fatigue degree can comprise: calculating a pulse peak-to-peak time period based on a pulse signal connected by a photoelectric sensor 12 in one of the left receiver and the right receiver; performing frequency spectrum analysis to a pulse signal within the pulse peak-to-peak time period to obtain frequency spectrum parameters; and, determining the fatigue degree based on the frequency spectrum parameters. According to the disclosure, the pulse peak-to-peak time period refers to an interval time between two adjacent peak values of a pulse signal.

In an embodiment of the disclosure, in a way of determining the fatigue degree, one photoelectric sensor 12 is only required to collect the pulse signal. It can be the photoelectric sensor 12 in the left receiver of the earphone and can also be the photoelectric sensor 12 in the right receiver of the earphone, which will not be defined in the embodiment of the disclosure. Based on one pulse signal, the pulse peak-to-peak time period of the pulse signal can be calculated. Further, frequency spectrum analysis can be performed to the pulse signal within the pulse peak-to-peak time period so as to obtain frequency spectrum parameters.

FIG. 5 is an example diagram of a power spectrum of a pulse signal within a pulse peak-to-peak time period. As shown in FIG. 5, within the pulse peak-to-peak time period, frequencies are mainly between 0.04˜0.4 Hz. In FIG. 5, 0.04˜0.15 Hz is regarded as low frequency (LF), and 0.15˜0.4 Hz is regarded as high frequency (HF). Certainly, division of the low frequency and the high frequency can be changed based on actual conditions. Power between 0.04˜0.4 Hz is called a total power (TP). The power corresponding to the low frequency 0.04˜0.15 Hz is regarded as low frequency (LF) power (that is, the low frequency component of the total power). The power corresponding to 0.15˜0.4 Hz is regarded as high frequency (HF) power (that is, the high frequency component of the total power). Generally, the LF power is a sign of a degree of sympathetic nerve excitability. The HF power is related to a mechanical change caused by breath and reflects a degree of parasympathetic nerve excitability. A ratio of the LF power to the HF power (i.e. LF power/the HF power) reflects balance of the sympathetic nerve and the parasympathetic nerve. The research shows that as the fatigue degree deepens, the LF power and the LF power/the HF power increase, and the HF power decreases remarkably. Thus, according to the disclosure, the LF power, the HF power, and the LF power/the HF power can be taken as the frequency spectrum parameters to determine the fatigue degree.

According to the disclosure, in order to determine a user's fatigue degree, it is required to obtain values of the frequency spectrum parameters of the user in a non-fatigue state when the user uses the earphone according to the disclosure for the first time (initially), and take them as standard values of the frequency spectrum parameters, so as to be compared with the subsequently obtained values of the frequency spectrum parameters. Thus, the method of determining the fatigue degree can comprise, compared to the standard values of the frequency spectrum parameters (in this example, a standard value of the LF power, the standard value of the LF power/a standard value of the HF power, the standard value of the HF power): when the LF power increases, the LF power/HF power increases, and the HF power decreases, it can be determined that the fatigue degree of the user increases; or, when the LF power increases, the LF power/the HF power increases, and the HF power keeps constant, it can be determined that the fatigue degree of the user increases; or, when the LF power keeps constant, the LF power/the HF power increases, and the HF power decreases, it can be determined that the fatigue degree of the user increases.

In another embodiment of the disclosure, the photoelectric sensors 12 collecting two pulse signals of the left and right ears comprises: collecting two pulse signals of the left and right ears in a non-fatigue state of a user. The processor 11 is used for calculating the pulse transmission time and the pulse peak-to-peak time period in the non-fatigue state based on the pulse signals in the non-fatigue state collected by the photoelectric sensors 12. In addition, the blood pressure value of the user in the non-fatigue state is measured using a blood pressure measurement device, and is stored in association with the pulse transmission time and the pulse peak-to-peak time period as previously obtained.

Thus, according to the disclosure, the physical sign detecting earphone 10 according to the disclosure can also calculate the blood pressure value measured in real time through a formula:

$$Δ BP = - \frac{2}{γ PTT} Δ PTT \qquad ( 2 )$$

In the above formula (2), PTT represents a pulse transmission time measured in real time, ARP represents a difference value between a blood pressure value in the non-fatigue state and a blood pressure value measured in real time, ΔPTT represents a difference value between the pulse transmission time in the non-fatigue state and the pulse transmission time measured in real time, γ represents an elastic coefficient of a blood vessel, and the blood pressure value in the non-fatigue state is measured by a blood pressure measurement device. The processor 11 can calculate the blood pressure value measured in real time through the above formula (2). The ΔPTT, PTT (which can be calculated after two pulse signals are measured in real time) and γ (which can also be fitted or counted according to the formula (1)) in the formula (2) are a known quantity. In addition, the blood pressure value of the user in to the non-fatigue state is also a known quantity, and ΔBP is the difference value between the blood pressure value in the non-fatigue state (which can be measured by a blood pressure measurement device) and the blood pressure value measured in real time (which needs to be finally calculated).

As described above, the processor 11 can obtain standard values of the frequency spectrum parameters in the non-fatigue state based on the pulse peak-to-peak time period in the non-fatigue state. Subsequently, after the processor 11 obtains the frequency spectrum parameters measured in real time based on the pulse signal collected by the photoelectric sensor 12 in one of the left receiver and the right receiver, values of the frequency spectrum parameters measured in real time can be compared with the above standard values of the frequency spectrum parameters to determine the fatigue degree. The way of determining the fatigue degree can be the same as that of the above embodiment of the disclosure. For example, when the LF power and the LF power/the HF power measured in real time increase compared to the standard values of the corresponding frequency spectrum parameters, and the HF power measured in real time decreases compared to the standard value of the corresponding frequency spectrum parameter, it can be determined that the fatigue degree of the user increases, i.e., the user is in a fatigue state.

Because a human body is a relative complex system, there is a great difference between different users. In order to eliminate individual difference, the one point calibration method can be used. That is, when a user uses the earphone according to the disclosure for the first time, two pulse signals of left and right ears of the user in a non-fatigue state of the user are collected by the earphone according to the disclosure, a blood pressure value of the user is measured by a blood pressure measurement device, and a pulse transmission time and a pulse peak-to-peak time period in the non-fatigue state are calculated according to the way in the above embodiments of the disclosure. Subsequently, the preset universal constant values can be adjusted according to the above formula (1) using the measured blood pressure value and the calculated pulse transmission time, so as to be adapted to the particular user. In addition, frequency spectrum parameters can be obtained based on the measured pulse peak-to-peak time period in the non-fatigue state of the particular user as standard values of the frequency spectrum parameters, so as to be compared with the subsequent frequency spectrum parameters measured in real time to obtain the fatigue degree of the particular user.

It should be noted that, in the embodiments of the disclosure, when the user is in the non-fatigue state, the two pulse signals of the left and right ear are collected, and the blood pressure value of the user in the non-fatigue state is measured by a blood pressure measurement device. The measurement is performed in the non-fatigue state for obtaining the standard blood pressure value and standard values of the frequency spectrum parameters so as to serve as reference values of subsequent measurements. Determination of the non-fatigue state can include pre-determination of physical sign information of a user, for example, making determination by formulating standards of physical sign information of users. When the measured standard blood pressure value and the standard values of the frequency spectrum parameters are within the formulated standards, it shows that the user is in the non-fatigue state at this time.

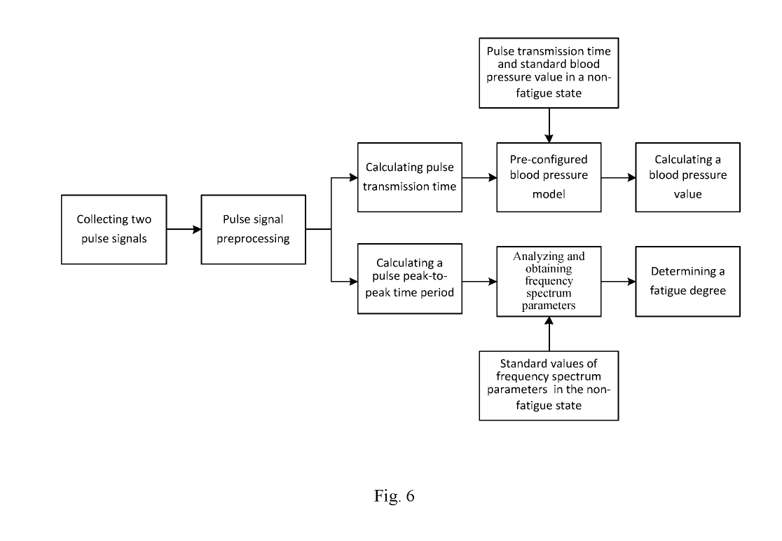

FIG. 6 is a schematic diagram of measurement principle of a physical sign detecting earphone provided by an embodiment of the disclosure. After two pulse signals are collected, the pulse signals can be preprocessed firstly, and then a blood pressure value and a fatigue degree can be calculated respectively in two ways. The calculating ways have been explained in detail in the above embodiments, which will not be repeated here. In the calculating process, for a particular user, a blood pressure value and frequency spectrum parameter standard values for the particular user in a non-fatigue state can be obtained in one point calibration way. Subsequently, a real time blood pressure value and/or a fatigue degree of the user can be calculated and determined based on the blood pressure value and/or the frequency spectrum parameter standard values of the user in the non-fatigue state.

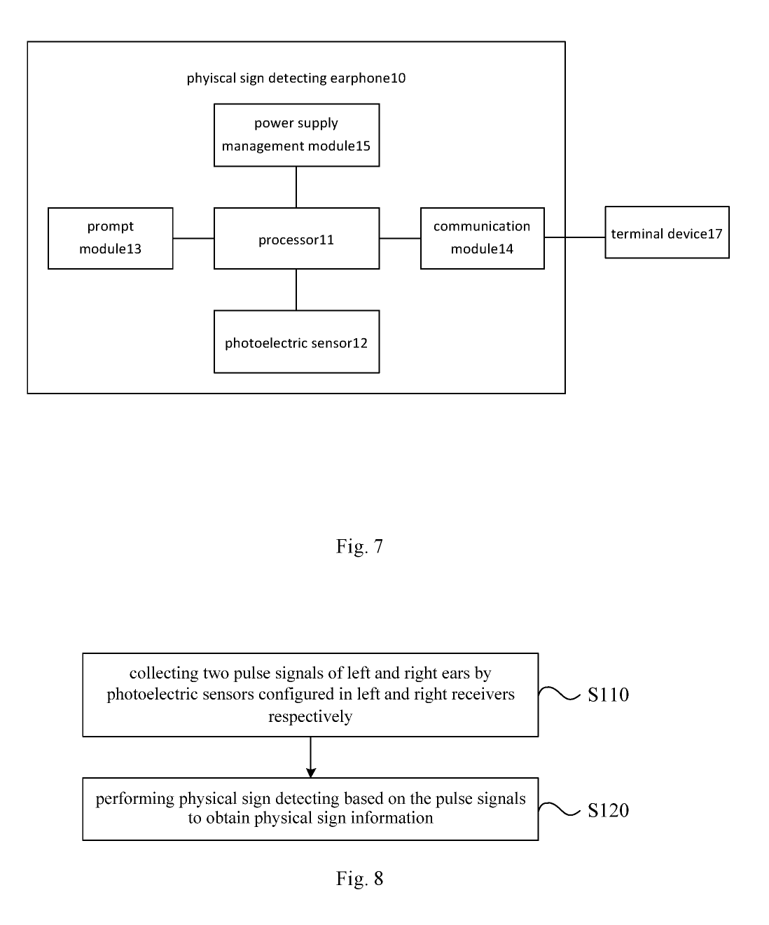

FIG. 7 is a schematic diagram of a module architecture of another physical sign detecting earphone provided by an embodiment of the disclosure. In addition to the processor 11 and the photoelectric sensors 12 shown in the above embodiments of the disclosure, the physical sign detecting earphone 10 as shown in FIG. 7 can further comprise: a prompt module 13 for prompting an abnormal state of physical sign information when the physical sign information obtained by the processor 11 is not conformity with a preset condition.

The physical sign information being not conformity with the preset condition can comprise one or more of: a blood pressure value being out of a preset blood pressure threshold range, and a time in which the user is in a fatigue state being longer than a preset time threshold.

In an embodiment of the disclosure, the prompt module 13 for example can be a miniature vibrator and is arranged at a position below the receivers of the earphone. Referring to the schematic diagram of the physical sign detecting earphone as shown in FIG. 2, FIG. 2 shows the position of the prompt module 13. When the processor 11 determines that the physical sign information of a user is not conformity with a preset condition, the user can be prompted by vibration of the miniature vibrator to adopt corresponding countermeasures. For example, the standard blood pressure value range is: 60 (mmHg)<diastolic pressure<90 mmHg, 90 mmHg<systolic pressure<140 mmHg. When a blood pressure value measured in real time is out of the above range, it is regarded that the user's blood pressure is abnormal. At this moment, the miniature vibrator prompts the user by vibration that the blood pressure value is abnormal. In addition, when it is determined that a time in which the user is in a fatigue state is longer than a preset time threshold (e.g., 30 minutes) by comparing with the standard values of the frequency spectrum parameters, the miniature vibrator prompts the user by vibration that he/she has been in the fatigue state for a relatively long time. In this embodiment, the processor 11 can start timing exactly when it is determined that the user is in the fatigue state. If, starting from the timing, the processor 11 continuously determines that the user is in the fatigue state and the recorded time is longer than the preset time threshold, the user will be prompted that he/she has been in the fatigue state for a relatively long time.

It should be noted that in actual applications, a vibration form for prompting an abnormal blood pressure value and a fatigue state can be set as different vibration modes, and a user can determine specific conditions of anomaly of physical sign information based on different vibration modes. In addition, the prompt module according to the disclosure can be any other forms such as flicker and so on that can be conceived by the skilled person in the art.

In an embodiment of the disclosure, the physical sign detecting earphone 10 can further comprise: a communication module 14 for transmitting a physical sign information obtained by the processor 11 to a terminal device so that the terminal device displays the physical sign information obtained in a preset time range.

In an embodiment of the disclosure, the physical sign detecting earphone 10 can further comprise: a power supply management module 15 for supplying power for components of the physical sign detecting earphone 10. It should be noted that the power supply management module 15 can also directly supply power only for some components therein (e.g., the processor 11), and in turn supply power for remaining components through these components. The embodiments of the disclosure will not define it specifically.

In an embodiment of the disclosure, the communication module 14 for example can be a Bluetooth module. It can communicate with a terminal device 17, and can transmit the obtained physical sign information to the terminal device 17 that establishes a connection with the Bluetooth module under instruction of the processor 11, so as to be displayed and stored by the terminal device 17. By obtaining or storing blood pressure values and the fatigue degrees of several consecutive days, the terminal device 17 can monitor a long-term trend of a physical state of a user, so as to provide the user with appropriate health care suggestions.

The processor 11 in an embodiment of the disclosure for example is a 32 bit lower power consumption reduced instruction set computing (RISC) microprocessor, e.g., an advanced RISC machine (ARM) processor. It can process the collected signals and control the prompt module 13 and the communication module 14, etc.

Based on the physical sign detecting earphone provided by the above embodiments of the disclosure, an embodiment of the disclosure further provides a physical sign detecting method. The physical sign detecting method is carried out by the physical sign detecting earphone 10 provided by any of the above embodiments of the disclosure.

FIG. 8 is a flow chart of a physical sign detecting method provided by an embodiment of the disclosure. The method provided by the embodiment can be carried out by any of the physical sign detecting earphones 10 as shown in FIGS. 1, 2, and 7. The method provided by the embodiment of the disclosure can comprise steps of: S110, collecting two pulse signals of left and right ears by photoelectric sensors configured in left and right receivers respectively; S120, performing physical sign detecting based on the pulse signals to obtain physical sign information.

The physical sign detecting method provided by the embodiment of the disclosure is an intelligent measurement performed to physical sign information of a human body. The operation of collecting two pulse signals of left and right ears can be performed by photoelectric sensors arranged in the left and right receivers respectively. In this way, when a user uses the physical sign detecting earphone to listen to music or watch videos, the pulse signals of the left and right ears of the user can be collected by the photoelectric sensors arranged in the left and right receivers, so as to detect the physical sign information of the user without using a particular measurement device. In an embodiment, the photoelectric sensors can also perform to the two collected pulse signals preprocessing operations such as signal amplification, analog-to-digital conversion, noise elimination, and so on. Subsequently, the processor performs physical sign detecting to the two collected or preprocessed pulse signals.

In an embodiment of the disclosure, the physical sign detecting can be performed based on the two collected pulse signals. Because the two pulse signals are pulse signals collected at different positions of a human body, and the distances between the different positions of the human body and a heart are different, transmission of the two pulse signals have different wave peaks and wave valleys. The physical sign information of the user can be calculated based on a difference between the two pulse signals. The physical sign information can reflect the physical state of the user.

In an embodiment of the disclosure, the physical sign information obtained in S120 can include but not limited to: one or more of a blood pressure value and a fatigue degree. In the current situation that the pace of life continues to speed up, the high blood pressure has become a major hidden danger that induces people's diseases and has severely harmed the people's health. In addition, under the high pace life and high intensity work, most of people has different levels of mental fatigue, which will also induce some diseases and harm people's health. Therefore, the physical sign information in the embodiment of the disclosure can be used for monitoring whether a user has a high blood pressure and whether a user is in the state of mental fatigue. It should be noted that the physical sign information in the embodiment of the disclosure can also include other indexes such as a heart rate value and so on that can reflect the physical state of the user.

The physical sign detecting method provided by an embodiment of the disclosure collects two pulse signals of left and right ears through photoelectric sensors configured in left and right receivers respectively and performs physical sign detecting by a processor based on the collected pulse signals so as to obtain the physical sign information which can reflect the physical state of the user of the earphone. The physical sign detecting method provided by an embodiment of the disclosure is easy to perform measurement and can perform measurement of the physical sign information when a user uses the earphone, thereby achieving the object of measuring the physical sign information of the user in real time and solving the problems that the blood pressure measurement and fatigue detection in the prior art have poor convenience and cannot be performed in real time.

The above embodiments of the disclosure have shown that the physical sign information can include: one or more of a blood pressure value and a fatigue degree.

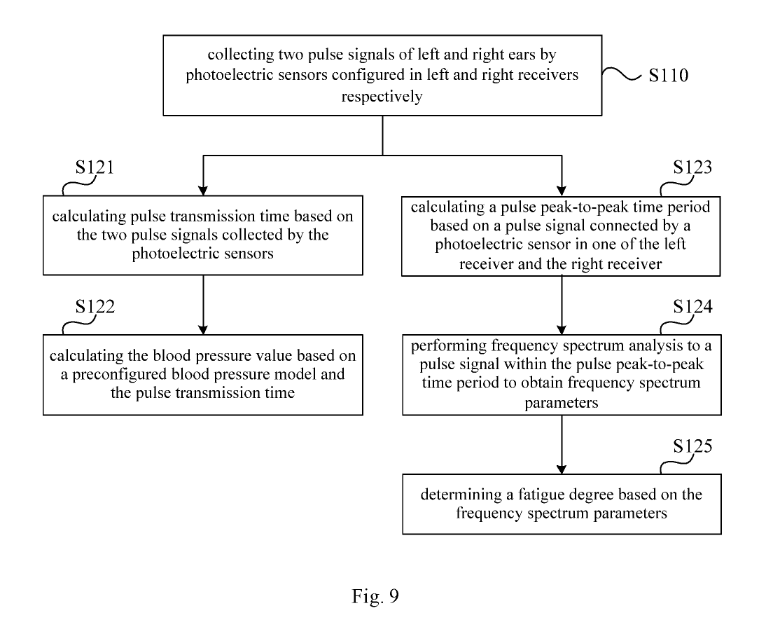

FIG. 9 is a flow chart of another physical sign detecting method provided by an embodiment of the disclosure. In FIG. 9, step S120 in FIG. 8 is further divided into different steps. Particularly, in FIG. 9, the physical sign detecting method can comprise:

S121, calculating a pulse transmission time based on the two pulse signals collected by the photoelectric sensors, the pulse transmission time being a time difference between peak values of the two pulse signals within a same electrocardio cycle;

S122, calculating the blood pressure value based on a preconfigured blood pressure model and the pulse transmission time.

In an embodiment of the disclosure, in the process of measuring the blood pressure value, it is required to firstly calculate the time difference between the peak values of two pulse signals within the same electrocardio cycle, i.e., the pulse transmission time. Reference can be made to the waveform diagram of transmission of pulse signals as shown in FIG. 3. The pulse transmission time can be obtained by comparing the time difference between the pulse wave peaks of the two pulse signals within the same electrocardio cycle in FIG. 3. Subsequently, a blood pressure value corresponding to the collected pulse signals, i.e., the current blood pressure value of the user, can be calculated by comparing the obtained pulse transmission time with a preconfigured blood pressure model.

In an actual application of the embodiment of the disclosure, obtaining the pre-configured blood pressure model can comprise a formula:

$$BP = \frac{1}{γ} [ ln ( \frac{ρ {dS}^{2}}{α {E}_{0}} ) - 2 ln PTT ] ; \qquad ( 1 ) .$$

In the above formula (1), as described above, BP represents a blood pressure value, PTT represents a pulse transmission time, γ represents an elastic coefficient of a blood vessel, p represents blood density, d represents a diameter of the blood vessel, S² represents a cross-sectional area of the blood vessel, α represents a thickness of the blood vessel, E₀ represents an initial vascular elasticity modulus.

In an embodiment of the disclosure, the physical sign detecting method as shown in FIG. 9 can comprise:

S123, calculating a pulse peak-to-peak time period based on a pulse signal connected by a photoelectric sensor in one of the left receiver and the right receiver;

S124, performing frequency spectrum analysis to the pulse signal within the pulse peak-to-peak time period to obtain frequency spectrum parameters; and

S125, determining a fatigue degree based on the frequency spectrum parameters.

In an embodiment of the disclosure, the above frequency spectrum parameters can include: a LF power, a HF power and the LF power/the HF power. Reference can be made to the schematic diagram of the power spectrum of a pulse signal within a pulse peak-to-peak time period as shown in FIG. 5, which shows the main frequency parameters such as LF, HF. The research shows that as the fatigue degree deepens, the LF power and the LF power/the HF power increase, and the HF power decreases remarkably. Therefore, the method of determining the fatigue degree can comprise, relative to standard values of the frequency spectrum parameters: when the LF power increases, the LF power/the HF power increases, and the HF power decreases, it can be determined that the fatigue degree of the user increases; or, when the LF power increases, the LF power/the HF power increases, and the HF power keeps constant, it can be determined that the fatigue degree of the user increases; or, when the LF power keeps constant, the LF power/the HF power increases, and the HF power decreases, it can be determined that the fatigue degree of the user increases.

In an embodiment of the disclosure, collecting the two pulse signals of the left and right ears in the physical sign detecting method comprises: collecting two pulse signals of left and right ears of a user in a non-fatigue state. In this case, the physical sign detecting method can comprise calculating a pulse transmission time and a pulse peak-to-peak time period in the non-fatigue state based on the pulse signals in the non-fatigue state collected by the photoelectric sensors 12. In addition, the blood pressure value of the user in the non-fatigue state is measured using a blood pressure measurement device, and is stored in association with the pulse transmission time and the pulse peak-to-peak time period as previously obtained.

Thus, according to the disclosure, the physical sign detecting method according to the disclosure can also calculate the blood pressure value measured in real time through a formula:

$$Δ BP = - \frac{2}{γ PTT} Δ PTT \qquad ( 2 )$$

In the above formula (2), PTT represents a pulse transmission time measured in real time, ΔBP represents a difference value between a blood pressure value in the non-fatigue state and a blood pressure value measured in real time, ΔPTT represents a difference value between the pulse transmission time in the non-fatigue state and the pulse transmission time measured in real time, γ represents an elastic coefficient of a blood vessel, the blood pressure value in the non-fatigue state is measured by a blood pressure measurement device.

The blood pressure value measured in real time can be calculated through the above formula (2). The ΔPTT, PTT (which can be calculated after two pulse signals are measured in real time) and γ (which can also be fitted or counted according to the formula (1)) in the formula (2) are all a known quantity. In addition, the blood pressure value of the user in the non-fatigue state is also a known quantity, and ΔBP is the difference value between the blood pressure value in the non-fatigue state (which can be measured by a blood pressure measurement device) and the blood pressure value measured in real time (a blood pressure value which needs to be finally calculated). Therefore, the blood pressure value measured in real time can be obtained according to the formula (2).

As described above, the physical sign detecting method can comprise obtaining the standard values of the frequency spectrum parameters in the non-fatigue state based on the pulse peak-to-peak time period in the non-fatigue state. In addition, the above physical sign detecting method can comprise, after obtaining, based on the pulse signal collected by the photoelectric sensor 12 in one of the left receiver and the right receiver, the frequency spectrum parameters measured in real time, comparing the frequency spectrum parameters measured in real time with the above standard values of the frequency spectrum parameters to determine the fatigue degree. The way of determining the fatigue degree can be the same as that of the above embodiments of the disclosure. For example, when the LF power and the LF power/the HF power measured in real time increase compared to standard values of the corresponding frequency spectrum parameters, and the HF power measured in real time decreases compared to the standard value of the corresponding frequency spectrum parameter, it can be determined that the fatigue degree of the user increases, i.e., the user is in a fatigue state.

Because the human body is a relative complex system, there is a great difference between different users. In order to eliminate individual difference, the one point calibration method can be used. That is, when a user uses the earphone according to the disclosure for the first time, two pulse signals of left and right ears of the user in the non-fatigue state of the user are collected using the earphone according to the disclosure, the blood pressure value of the user is measured by a blood pressure measurement device, and the pulse transmission time and pulse peak-to-peak time period in the non-fatigue state are calculated according to the ways in the above embodiments of the disclosure. Subsequently, the preset universal constant parameter values can be adjusted according to the above formula (1) using the measured blood pressure value and the calculated pulse transmission time, so as to be adapted to the particular user. In addition, the frequency spectrum parameters can be obtained based on the measured pulse peak-to-peak time period in the non-fatigue state of the particular user as the standard values of the frequency spectrum parameters, so as to be compared with the subsequent frequency spectrum parameters measured in real time to obtain the fatigue degree of the particular user.

It should be noted that the embodiments of the disclosure collect the two pulse signals of the left and right ears when the user is in the non-fatigue state and measures the blood pressure value of the user in the non-fatigue state through a blood pressure measurement device. The measurement is performed in the non-fatigue state for obtaining the standard blood pressure value and the frequency spectrum parameter standard values so as to serve as the reference values of the subsequent measurements. The determination of the non-fatigue state can include pre-determination of the physical sign information of the user, for example, making determination by formulating standards of physical sign information of users. When the measured standard blood pressure value and the frequency spectrum parameter standard values are within the formulated standards, it shows that the user is in the non-fatigue state at this time.

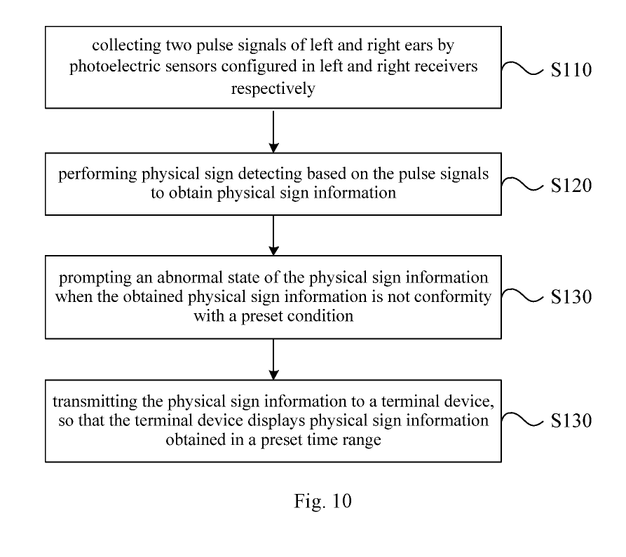

FIG. 10 is a flow chart of a yet another physical sign detecting method provided by an embodiment of the disclosure. In addition to the steps as shown in FIG. 8, the physical sign detecting method as shown in FIG. 10 can further comprise:

S130, prompting an abnormal state of the physical sign information when the obtained physical sign information is not conformity with a preset condition.

The physical sign information being not conformity with a preset condition can comprise one or more of: the blood pressure value being out of a preset blood pressure threshold range; and a time in which the user is in a fatigue state being longer than a preset time threshold.

In an embodiment of the disclosure, the method can further comprise: S140, transmitting the physical sign information to a terminal device, so that the terminal device displays physical sign information obtained in a preset time range.

In an embodiment of the disclosure, the terminal device can monitor a long-term trend of the physical sign information. Based on the blood pressure values and the fatigue degrees measured in several consecutive days, the terminal device can provide the user with appropriate health care suggestions. In addition, according to the disclosure, the terminal device can also perform any other appropriate processing to the physical sign information as needed.

Although the implementations disclosed by the disclosure have been described above, the contents are merely implementations used for convenient understanding of the disclosure rather than limiting the disclosure. Any skilled person in the art of the disclosure, on the premise of not departing from the spirit and the scope disclosed by the disclosure, can make any amendment and variation to forms and details of implementations. However, the patent protection scope of the disclosure still has to be subject to the scope defined by the attached claims.

1. A physical sign detecting earphone, comprising: a processor, and photoelectric sensors configured in left and right receivers respectively;
the photoelectric sensors are used for collecting two pulse signals of left and right ears;
the processor is used for performing physical sign detecting based on the pulse signals collected by the photoelectric sensors to obtain physical sign information.

2. The physical sign detecting earphone according to claim 1, wherein the physical sign information comprises: one or more of a blood pressure value and a fatigue degree.

3. The physical sign detecting earphone according to claim 2, wherein the processor performing physical sign detecting to obtain a blood pressure value comprises:
calculating pulse transmission time based on the two pulse signals collected by the photoelectric sensors, the pulse transmission time being a time difference between peak values of the two pulse signals within a same electrocardio cycle;
calculating the blood pressure value based on a preconfigured blood pressure model and the pulse transmission time.

4. The physical sign detecting earphone according to claim 3, wherein the preconfigured blood pressure model comprises a formula:
$$BP = \frac{1}{γ} [ ln ( \frac{ρ {dS}^{2}}{α {E}_{0}} ) - 2 ln PTT ] ;$$
wherein the BP represents the blood pressure value, the PTT represents the pulse transmission time, the γ represents an elastic coefficient of a blood vessel, the ρ represents blood density, the d represents a diameter of the blood vessel, the S² represents a cross-sectional area of the blood vessel, the α represents a thickness of the blood vessel, and the E₀ represents an initial vascular elasticity modulus.

5. The physical sign detecting earphone according to claim 2, wherein the photoelectric sensors are used for collecting two pulse signals of left and right ears in a non-fatigue state of a user when the physical sign detecting earphone is initially used, and collecting two pulse signals of left and right ears in real time;
the processor is used for calculating a pulse transmission time in the non-fatigue state based on the pulse signals in the non-fatigue state collected by the photoelectric sensors, calculating a pulse transmission time based on the two pulse signals connected by the photoelectric sensors in real time, and calculating a real time blood pressure value through a formula:
$$Δ BP = - \frac{2}{γ PTT} Δ PTT ;$$
wherein the PTT represents a pulse transmission time measured in real time, the ΔBP represents a difference value between a blood pressure value in the non-fatigue state and a blood pressure value measured in real time, the ΔPTT represents a difference value between the pulse transmission time in the non-fatigue state and the pulse transmission time measured in real time, the γ represents an elastic coefficient of a blood vessel, the blood pressure value in the non-fatigue state is measured by a blood pressure measurement device.

6. The physical sign detecting earphone according to claim 2, wherein the processor performing physical sign detecting to obtain a fatigue degree comprises:
calculating a pulse peak-to-peak time period based on a pulse signal connected by a photoelectric sensor in one of the left receiver and the right receiver;
performing frequency spectrum analysis to a pulse signal within the pulse peak-to-peak time period to obtain frequency spectrum parameters; and,
determining the fatigue degree based on the frequency spectrum parameters.

7. The physical sign detecting earphone according to claim 6, wherein the frequency spectrum parameters comprise: a low frequency power, a high frequency power and a ratio of the low frequency power to the high frequency power;
determining the fatigue degree based on the frequency spectrum parameters comprises: compared to standard values of the frequency spectrum parameters,
when the low frequency power increases, the ratio of the low frequency power to the high frequency power increases, and the high frequency power decreases, determining that the fatigue degree increases; or,
when the low frequency power increases, the ratio of the low frequency power to the high frequency power increases, and the high frequency power is constant, determining that the fatigue degree increases; or,
when the low frequency power is constant, the ratio of the low frequency power to the high frequency power increases, and the high frequency power decreases, determining that the fatigue degree increases.

8. The physical sign detecting earphone according to claim 7, wherein the standard values of the frequency spectrum parameters are obtained by:
the photoelectric sensor in one of the left receiver and the right receiver collecting a pulse signal in a non-fatigue state; and
the processor calculating a pulse peak-to-peak time period in the non-fatigue state based on the pulse signal in the non-fatigue state collected by the photoelectric sensor, and performing frequency spectrum analysis to the pulse signal within the pulse peak-to-peak time period to obtain values of frequency spectrum parameters as standard values of the frequency spectrum parameters.

9. The physical sign detecting earphone according to claim 1, further comprising: a prompt module for prompting an abnormal state of the physical sign information when the physical sign information obtained by the processor is not conformity with a preset condition.

10. The physical sign detecting earphone according to claim 9, wherein the physical sign information being not conformity with a preset condition comprises one or more of:
the blood pressure value being out of a preset blood pressure threshold range; and
a time in which a user is in a fatigue state being longer than a preset time threshold.

11. The physical sign detecting earphone according to claim 1, further comprising: a communication module for transmitting the physical sign information to a terminal.

12. A physical sign detecting method, comprising:
collecting, by photoelectric sensors configured in left and right receivers of a physical sign detecting earphone respectively, two pulse signals of left and right ears;
performing physical sign detecting based on the pulse signals to obtain physical sign information.

13. The physical sign detecting method according to claim 12, wherein the physical sign information comprises: one or more of a blood pressure value and a fatigue degree.

14. The physical sign detecting method according to claim 13, wherein performing physical sign detecting based on the pulse signals to obtain a blood pressure value comprises:
calculating a pulse transmission time based on the two pulse signals collected by the photoelectric sensors, the pulse transmission time being a time difference between peak values of the two pulse signals within a same electrocardio cycle;
calculating the blood pressure value based on a preconfigured blood pressure model and the pulse transmission time.

15. The physical sign detecting method according to claim 14, wherein the preconfigured blood pressure model comprises a formula:
$$BP = \frac{1}{γ} [ ln ( \frac{ρ {dS}^{2}}{α {E}_{0}} ) - 2 ln PTT ] ;$$
wherein the BP represents the blood pressure value, the PTT represents the pulse transmission time, the γ represents an elastic coefficient of a blood vessel, the ρ represents blood density, the d represents a diameter of the blood vessel, the S² represents a cross-sectional area of the blood vessel, the α represents a thickness of the blood vessel, and the E₀ represents an initial vascular elasticity modulus.

16. The physical sign detecting method according to claim 13, further comprising:
collecting two pulse signals of left and right ears in a non-fatigue state of a user by the photoelectric sensors when the physical sign detecting earphone is initially used;
calculating a pulse transmission time in the non-fatigue state based on the two pulse signals in the non-fatigue state;
collecting two pulse signals of left and right ears by the photoelectric sensors in real time;
calculating a pulse transmission time based on the two pulse signals connected by the photoelectric sensors in real time, and calculating a real time blood pressure value through a formula:
$$Δ BP = - \frac{2}{γ PTT} Δ PTT ;$$
wherein the PTT represents a pulse transmission time measured in real time, the ΔBP represents a difference value between a blood pressure value in the non-fatigue state and a blood pressure value measured in real time, the ΔPTT represents a difference value between the pulse transmission time in the non-fatigue state and the pulse transmission time measured in real time, the γ represents an elastic coefficient of a blood vessel, the blood pressure value in the non-fatigue state is measured by a blood pressure measurement device.

17. The physical sign detecting method according to claim 13, wherein performing physical sign detecting based on the pulse signals to obtain a fatigue degree comprises:
calculating a pulse peak-to-peak time period based on a pulse signal connected by a photoelectric sensor in one of the left receiver and the right receiver;
performing frequency spectrum analysis to a pulse signal within the pulse peak-to-peak time period to obtain frequency spectrum parameters; and
determining the fatigue degree based on the frequency spectrum parameters.

18. The physical sign detecting method according to claim 16, wherein the frequency spectrum parameters comprises: a low frequency power, a high frequency power and a ratio of the low frequency power to the high frequency power;
determining the fatigue degree based on the frequency spectrum parameters comprises: compared to standard values of the frequency spectrum parameters,
when the low frequency power increases, the ratio of the low frequency power to the high frequency power increases, and the high frequency power decreases, determining that the fatigue degree increases; or,
when the low frequency power increases, the ratio of the low frequency power to the high frequency power increases, and the high frequency power is constant, determining that the fatigue degree increases; or,
when the low frequency power is constant, the ratio of the low frequency power to the high frequency power increases, and the high frequency power decreases, determining that the fatigue degree increases.

19. The physical sign detecting method according to claim 12, further comprising:
prompting an abnormal state of the physical sign information when the obtained physical sign information is not conformity with a preset condition; and wherein the physical sign information being not conformity with a preset condition comprises one or more of:
the blood pressure value being out of a preset blood pressure threshold range; and
a time in which a user is in a fatigue state being longer than a preset time threshold.

20. The physical sign detecting method according to claim 12, further comprising: transmitting the physical sign information to a terminal.
